SEQUENCE LISTING (1 sequence)

SEQ ID NO: 1 - protein, 37 aa, Homo sapiens
LLGDFFRKSKEKIGKEFKRIVQRIKDFLRNLVPRTES

(19) United States
(12) Patent Application Publication
(10) Pub. No.: US 2016/0120938 A1
(43) Pub. Date: May 5, 2016

(54) P2X7 RECEPTOR AGONIST FOR USE IN PREVENTING OR TREATING KIDNEY INJURY

(71) Applicant: UCL BUSINESS PLC, London (GB)

(72) Inventors: Rajiv JALAN, London (GB); Danielle Motunrayo ADEBAYO, London (GB)

(73) Assignee: UCL BUSINESS PLC, London (GB)

(21) Appl. No.: 14/786,630

(22) PCT Filed: Apr. 23, 2014
(86) PCT No.: PCT/GB2014/051261

§ 371 Date: Oct. 23, 2015

(30) Foreign Application Priority Data
Apr. 23, 2013 (GB) 1307310.1

(51) Int. Cl. A61K 38/17; G01N 33/50; A61K 31/7076

(52) U.S. Cl. CPC A61K 38/1709; A61K 31/7076; G01N 33/5044; G01N 33/502; G01N 33/5088; G01N 2333/705; G01N 2800/347

(57) ABSTRACT

The present invention relates to the treatment or prevention of kidney injury and in particular acute kidney injury, such as acute kidney injury associated with cirrhosis. The invention relates to the treatment or prevention of such injury by administration of an agonist of the P2X7 receptor.

### FIELD OF THE INVENTION

The present invention relates to the treatment of dysfunction in the liver and kidneys. In particular, the present invention relates to methods that utilise agonists of P2X7R in order to prevent or treat kidney injury. The present invention also relates to the identification of P2X7R agonists that may be used in the treatment or prevention of such kidney injury.

### BACKGROUND TO THE INVENTION

Renal failure and renal dysfunction are frequently presenting complications in patients with cirrhosis or liver failure and are associated with high morbidity and mortality rates. Renal failure is characterised by a rapid deterioration in kidney function and is usually fatal unless a liver transplant is performed, although various treatments, such as dialysis, can prevent advancement of the condition.

Renal dysfunction and renal failure can affect individuals with cirrhosis (regardless of cause), severe alcoholic hepatitis, or fulminant hepatic failure, and usually occurs when liver function deteriorates rapidly because of an acute injury. Renal dysfunction in cirrhosis may be due to superimposed infection and inflammation or in its absence, a situation referred to as hepatorenal syndrome (HRS). These are relatively common complications of cirrhosis, occurring in 18% of cirrhotics within one year of their diagnosis, and in 39% of cirrhotics within five years of their diagnosis.

Cirrhotic patients who develop functional renal dysfunction present a challenge for current medical management strategies. Untreated, these patients have a 3-month mortality rate approaching 90%. With the best currently available treatment options, renal function is only improved in about 40% of these patients. Hence, there is an urgent need to understand better the pathophysiological basis of cirrhosis-related renal dysfunction, so as to develop new and effective treatment strategies.

Kidney injury such as acute kidney injury may therefore be associated with cirrhosis or liver failure. It may also occur in patients not having cirrhosis or liver failure, such as in association with sepsis, trauma, surgery or cancer.

### SUMMARY OF THE INVENTION

It has previously been suggested that signalling through the P2X7 receptor (P2X7R) leads to injury and inflammation. The Inventors have confirmed that expression of P2X7R protein was increased in the renal tubules and worsened with injury. However, the Inventors unexpectedly found that inhibition of P2X7R led to a worsening of renal function and that agonism of P2X7R had a protective effect.

Accordingly, the invention provides an agonist of P2X7 receptors (P2X7R) for use in a method of treating or preventing kidney injury, such as acute kidney injury.

The individual may be suffering from cirrhosis, acute on chronic liver failure or acute liver failure. The individual may be suffering from acute kidney injury or may be at risk of acute kidney injury. The individual may be suffering from sepsis, trauma or cancer. The individual may have undergone surgery or may be about to undergo surgery.

The P2X7R agonist may be used to treat or prevent kidney injury, such as acute kidney injury in an individual having cirrhosis. The P2X7R agonist may lead to increased expression of P2X7R in the kidney of the individual; and/or increased levels of P2X7R in the kidney of the individual; and/or increased P2X7R activity such as signalling via P2X7R in the kidney of the individual. The agonist may be a specific agonist of P2X7R.

Thus, the present invention also relates to a method of treating or preventing kidney disease or kidney injury in an individual in need thereof, said method comprising a step of administering to said individual an agonist of P2X7R. Similarly, the present invention also relates to the use of an P2X7R agonist in the manufacture of a medicament for use in the treatment of an individual suffering from or at risk of kidney disease or kidney injury.

In any of the methods and uses described herein, the kidney injury may be acute kidney injury.

The invention also provides a method of identifying an agent suitable for use in treating or preventing kidney disease or kidney injury, the method comprising determining whether a test agent is capable of increasing the amount of P2X7R activity such as the amount of signalling via P2X7R, wherein the ability to increase the amount or such P2X7R activity indicates that the compound may be suitable for use in treating or preventing kidney disease or kidney injury. Such a method may be carried out by assessing the amount of P2X7R activity such as signalling via P2X7R in cells or a tissue that expresses or contains P2X7R. For example, the amount or activity of P2X7R may be assessed in the kidney or in tissue or cells derived from the kidney; or in another cell or tissue that expresses P2X7R. Such a method may be carried out by administering a test agent to a bile duct ligated rat and determining whether the presence of the test agent leads to an increase in the amount or activity of P2X7R in the kidney of the rat.

### BRIEF DESCRIPTION OF THE DRAWINGS

FIG. 1A summarises the spectrum of hepatorenal disease in patients with advanced cirrhosis. AKI=acute kidney injury; HRS=hepatorenal syndrome, KD=kidney disease; CKD=chronic kidney disease. FIG. 1B summarises the pathophysiology of hepatorenal syndrome, particularly where there is an acute precipitating event. Taken from Wong et al Gut 2011; 60: 702-709.

FIG. 2 summarises the action of the P2X7 receptor. Taken from Arulumaran et al Exp Opin Investig Drugs, 2011; 20: 897-915.

FIG. 3 shows immunohistochemistry showing P2X7 receptor expression in kidneys from a cirrhotic AKI model. A=sham treated animal, B=BDL (bile duct ligated) animal; C=BDL+lipopolysaccharide (LPS); D=BDL+P2X7R antagonist; E=BDL+P2X7R antagonist+LPS.

FIG. 4 shows the effects of P2X7R antagonist on (A) urine NGAL levels and (B) creatinine levels.

FIG. 5 shows the effects of P2X7R antagonist on (A) plasma endotoxin levels in a BDL model; (B) plasma TNFα levels; and (C) ALT in a cirrhotic AKI model.

FIG. 6 shows P2X7 receptor expression in kidney homogenates.

FIG. 7 shows the effects on creatinine levels of renal administration of BzATP.

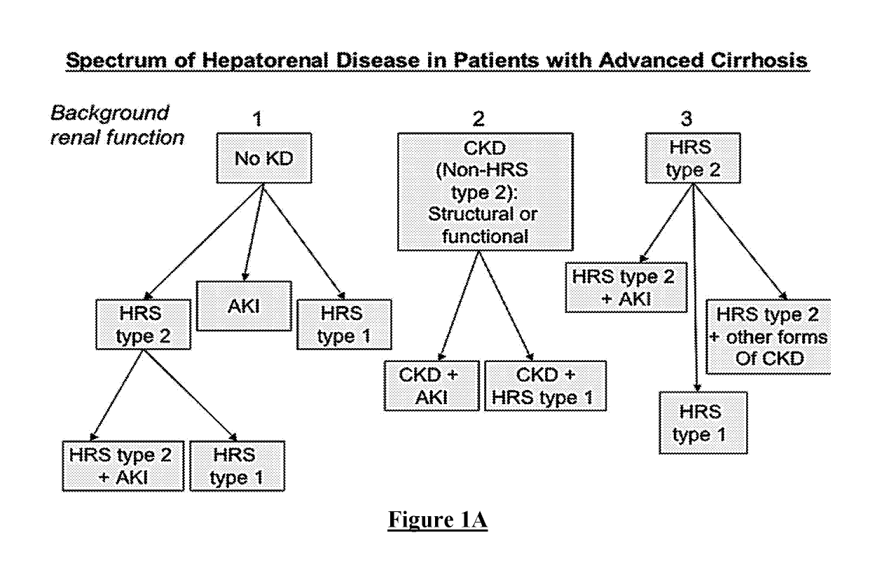

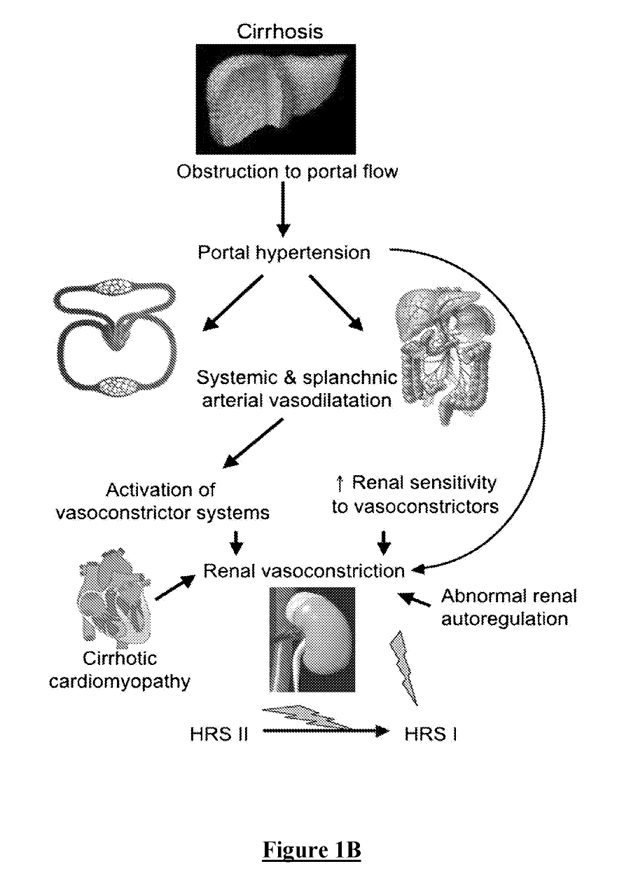

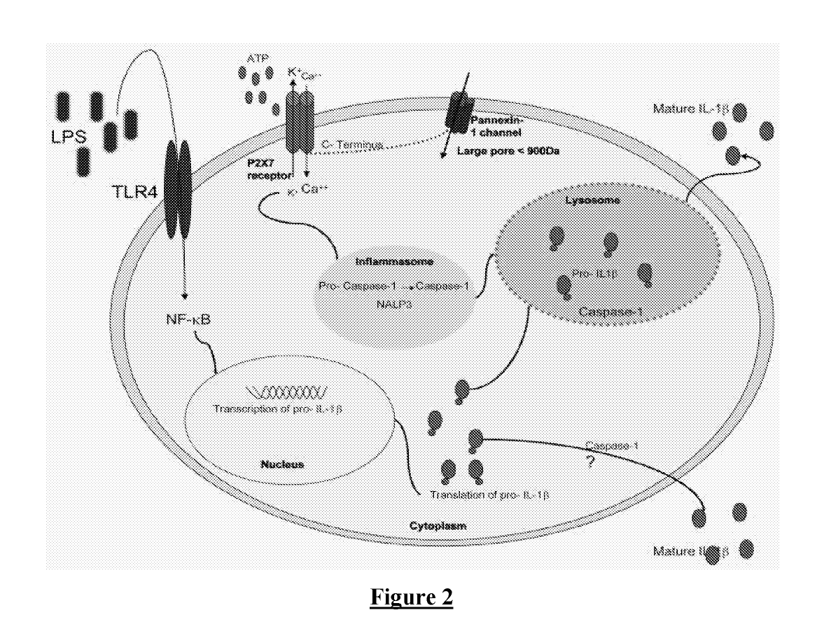

### DETAILED DESCRIPTION OF THE INVENTION

It was first proposed in 1970 that intracellular adenosine triphosphate (ATP) might have an additional role outside cells as a signaling molecule. The role of ATP as an extracellular messenger in purinergic cell signalling has been investigated extensively since that time.

The purinergic receptors can be classified into P1 receptors (acted on by adenosine) and P2 receptors (acted on by ATP and its breakdown products ADP and AMP). The P2 receptors have been further sub-classified as P2X receptors (inotropic ligand-gated non-selective cation channel receptors) and P2Y receptors (G protein coupled receptors). Further subtypes exist within the P2X and P2Y receptor groups.

The P2X7 receptor (P2X7R) is a distinct member of the P2X group of receptors. Its downstream signalling is coupled to pro-inflammatory cascades. It is a 595 amino acid polypeptide with two membrane spanning domains and has a long intracellular C terminus compared with other P2X receptors (North, Physiol Rev 2002; 82:1013-67). The P2X7R is expressed on many types of cells, the most studied being macrophages and monocytes, and has been proposed to play a role in regulating cell survival and the extracellular release of IL-1β and IL-18, which act as key mediators of the host inflammatory response to infection. The expression and activity of the P2X7R is significantly up regulated in macrophages in response to inflammatory stimuli, including TNFα and lipopolysaccharide (LPS). As well as release of mature IL-1β and IL-18, prolonged activation of the receptor results in irreversible pore formation and cell death by apoptosis or necrosis.

In normal rat kidneys, there is a low level of P2X7R, localised to the glomeruli. However in nephropathy models of diabetes, hypertension and glomerulonephritis, the expression of the receptor is upregulated. In addition, treatment with a P2X7R antagonist reduces the severity of glomerular injury in an animal model of glomerulonephritis.

The inventors found that protein expression of P2X7R was increased in the renal tubules which worsened with superimposed injury in LPS treated rats. However, inhibition of P2X7R unexpectedly further worsened renal function in these animals. The inventors also found that an agonist of P2X7R protected the kidneys. The present invention is based on these unexpected findings, which lead to the use of agonists of P2X7R agents for the prevention or treatment of kidney injury, such as acute kidney injury.

### P2X7R Agonists

The present invention relates to the agonism of P2X7 receptors (P2X7R). An agonist of P2X7R may be any compound or molecule that promotes or increases the activity, function or amount of P2X7R. Preferably the agonist functions in cells, tissues or organs that express P2X7R such as in the kidney of the patient. The agonist may act preferentially in the kidney or may act at a number of locations including the kidney. The agonist may act preferentially in particular cell types such as inflammatory cells, platelets or neurons. Preferably the agonist leads to an increase in P2X7R activity, function or amount in the cells, tissues or organs of an individual to whom the agonist is administered, such as in one of more of the liver, kidneys, brain, and the heart of the individual. The increase in activity may be an increase in signalling via P2X7R. The agonist may be targeted to the kidney or other organs, cells or tissues such as those listed above during administration as discussed further below.

Preferred agonists are those that increase the activity (e.g. signalling via P2X7R) or amount (e.g. expression level measured as mRNA or protein level) of P2X7R by at least 10%, at least 20%, at least 30%, at least 40%, at least 50%, at least 60%, at least 70%, at least 80% or at least 90% compared to the amount seen in the absence of the agonist. For example, increases of these sizes may be seen in the kidney or kidney tissue of a subject to whom the agonist has been administered. Increases of these sizes may be seen in other tissues or organs of the individual, such as in the liver and/or heart of the individual.

The agonist may act specifically or selectively to antagonise P2X7R. The terms specific and selective are used interchangeably herein to refer to an effect in relation to P2X7R in preference to an effect on other purinergic receptors, such as other P2 receptors or other P2X receptors. That is, the effect of the agonist on P2X7R may be greater than any other biological effect of the agonist. Such an agonist may be specific to the agonism of P2X7R, that is it may increase the activity of P2X7R, but not other purinergic receptors as discussed herein. By activity of P2X7R herein is meant, for example, signalling via P2X7R. Such an agonist may additionally or alternatively be specific to the expression of P2X7R, that is it may increase the expression of P2X7R but not other purinergic receptors such as those discussed herein.

A specific agonist for use in accordance with the present invention may be an agonist of P2X7R as described herein, that does not act as an agonist of other purinergic receptor types such as other P2 receptors or other types of P2X receptor. A specific agonist for use in accordance with the present invention may act on P2X7R in preference to other purinergic receptor types. For example, an agonist of P2X7R for use in accordance with the present invention may have one or more of the characteristics of an P2X7R agonist as described herein, such as this ability to increase P2X7R expression or signalling via P2X7R, but may not have such characteristics in relation to other purinergic receptor types, or may have such characteristics to a lower level in relation to other purinergic receptor types when compared to P2X7R. For example, an agonist that increases the activity (e.g. signalling via that receptor) or expression of P2X7R may not increase the activity (e.g. signalling via that receptor) or expression of one or more other purinergic receptor types, or may increase the activity of the other purinergic receptor type(s) to a lesser extent than for P2X7R. The lesser extent may be measured as, for example, a lower percentage increase when compared to the activity or expression in the absence of the agonist, such as an increase in activity or expression of less than 30%, less than 20%, less than 10%, less than 5%, less than 2%, less than 1% or less than 0.5% when compared to the activity or expression in the absence of the agonist or when compared to its effect on P2X7R. An agonist that increases the activity, expression or amount of P2X7R may therefore not increase the expression or amount of other purinergic receptor types, or may increase the expression of other purinergic receptor types to a lesser extent, such as a lower percentage increase, than its effect on P2X7R.

By other purinergic receptor types herein is meant any purinergic receptor that is not P2X7R. For example, the other purinergic receptor type may be another type of P2 purinergic receptor or another type of P2X receptor.

The other purinergic receptor may be more than one of these purinergic receptor types. For example, the effects of the agonist on an P2X7 receptor may be specific, as discussed above, when compared to the effects of that agent on other P2 receptors, when compared to the effects of that agent on other P2X receptors, and/or when compared to the effects of that agent when compared to other purinergic receptors that are not P2 receptors or are not P2X receptors. The effects of the agonist on a P2X7 receptor may be specific as discussed above when compared to all other classes of purinergic receptor that are present.

The specificity of the P2X7R agonist may apply within the whole body of the individual to be treated, that is the actions of the P2X7R agonist may be specific as discussed above throughout the body of the individual. The specificity of the P2X7R agonist may apply within particular tissues of the individual, such as the liver, kidneys or heart. That is, in one embodiment, the P2X7R agonist may act specifically to agonise P2X7R as discussed above within the kidney of the individual being treated. This may be achieved by administering or targeting the P2X7R agonist to the organ or tissues of interest, such as the kidney.

The P2X7R agonist may therefore be a specific agonist of P2X7R as described above. For example, the P2X7R agonist may not be an agonist of other types of P2 or other types of P2X receptor.

Any agent capable of promoting or increasing the activity or function of P2X7R may be suitable for use in the methods of the present invention. Agonists for use in accordance with the present invention may be direct or indirect agonists of P2X7R.

Direct agonists are agents whose activity is directly on P2X7R. For example, direct agonists may be agents that act directly on the P2X7 receptor to increase its activity. A direct agonist may be an agent that promotes P2X7R function or that stabilises the P2X7 receptor. A direct agonist may increase the amount of P2X7R by providing additional P2X7R molecules or improving the stability of P2X7R molecules within the patient. A direct agonist may be an agent that acts on the P2X7R gene, promoter or other gene regulatory regions to increase expression of the P2X7R. A direct agonist may be an enhancer, transcription factor, microRNA or microRNA blocker such as antisense molecule that increases expression of P2X7R. A direct agonist may increase expression of P2X7R by promoting or increasing expression from the endogenous P2X7R gene. A direct agonist may itself provide further copies of the P2X7R gene to a cell, or may provide P2X7R expression products such as RNA or protein molecules. For example, a direct agonist may be a vector or plasmid that is capable of expressing P2X7R within a cell, such as a vector that is capable of incorporation into the genome of such a cell.

Indirect agonists are agents whose activity is not directly on P2X7R. For example, indirect agonists may be agents that act to increase extracellular ATP in the region of the P2X7R, such as in the kidney of the individual to be treated. This may be achieved by targeting the indirect agent to the tissue or organ of interest, such as the kidney. For example, the agonist may be administered direct into the kidney, such as by intrarenal administration.

Any agent or molecule having the properties described above may be used as an P2X7R agonist in accordance with the present invention. Examples of P2X7R agonists that may be used in accordance with the present invention include:

Benzoyl benzoyl-ATP (BzATP), 2′(3′)-O-(4-Benzoylbenzoyl)adenosine-5′-triphosphate.

KN-62; 4-[(2S)-2-[(5-isoquinolinylsulfonyl-)methylamino]-3-oxo-3-(4-phenyl-1-piperazinyl)prop-yl]phenyl isoquinolinesulfonic acid ester

human cathelicidin LL-37, a peptide having the sequence

LLGDFFRKSKEKIGKEFKRIVQRIKDFLRNLVPRTES

Further molecules as described in Baraldi et al Curr Top Med Chem, 2004; 4:1707-17.

The P2X7R agonist may be a molecule that is capable of binding to, and promoting the activity of P2X7R.

The P2X7R agonist may be a small molecule, chemical, peptide, microRNA or polynucleotide.

The P2X7R agonist may act to increase the expression of P2X7R either from the endogenous gene or by introducing additional copies of the P2X7R gene to the patient of interest.

The terms “nucleic acid molecule” and “polynucleotide” are used interchangeably herein and refer to a polymeric form of nucleotides of any length, either deoxyribonucleotides or ribonucleotides, or analogs thereof. A nucleic acid may comprise conventional bases, sugar residues and inter-nucleotide linkages, but may also comprise modified bases, modified sugar residues or modified linkages. A nucleic acid molecule may be single stranded or double stranded.

Any of the agonists described herein may therefore be used to agonise P2X7R, i.e. to increase the amount of P2X7R that is present, and/or the activity (e.g. signalling via) or the function of the P2X7R. This agonism may take place in any location or tissue where P2X7R is present. The agonism may take place in one or more organs selected from the brain, kidney, liver and heart. The agonism may take place on cells expressing P2X7R, such as inflammatory cells, platelets and/or neurons. Preferably these agonising effects take place in the kidney.

An agonist of P2X7R may be an agent that increases the production of endogenous P2X7R. For example, the agent may act within the cells of the subject to promote or maintain the expression of P2X7R. Such an agent may be a transcription factor, microRNA or enhancer that acts on the P2X7R gene to promote or maintain gene expression.

### Screening Methods

The present invention also provides methods for the identification of agents suitable for use in the treatment of kidney injury. For example, the invention provides methods for the identification of agonists of P2X7R which are suitable for use in treating or preventing kidney injury, such as acute kidney injury. Agonists identified by this method may be agonists of P2X7R having any of the characteristics or effects described above. Agonists identified by the methods described herein may be suitable for use in the treatment or prevention of kidney injury or in the treatment or prevention of any of the conditions or symptoms described herein.

Accordingly, the invention provides a method of identifying an agent for use in the treatment or prevention of kidney injury, the method comprising determining whether a test agent is capable of increasing the activity or expression of P2X7R. For example, the method may involve determining whether a test agent is capable of increasing the amount or activity of P2X7R, wherein the ability to increase the amount or activity of P2X7R indicates that the compound may be suitable for use in treating or preventing kidney injury as described herein.

The method may comprise assessing the amount or activity of P2X7R in a particular cell or tissue type. This may be any cell of tissue that expresses P2X7R. For example, the method may assess the amount or activity of P2X7R in the kidney or in tissue or cells derived from the kidney; in the liver or heart or cells derived from the liver or heart; in inflammatory cells, platelets or neurons; or in any other cell or tissue that expresses P2X7R.

A test agent for use in a screening method of the invention refers to any compound, molecule or agent that may potentially agonise P2X7R. The test agent may be, or may comprise, for example, a peptide, polypeptide, protein, polynucleotide, microRNA, small molecule or other compound that may be designed through rational drug design starting from known agonists of P2X7R.

The test agent may be any agent having one or more characteristics of an agonist of P2X7R as described above.

The test agent to be screened could be derived or synthesised from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g. phage display) libraries. Multiple test agents may be screened using a method of the invention in order to identify one or more agents having a suitable effect on P2X7R, such as promotion of P2X7R activity or expression.

The screening methods of the invention may be carried out in vivo, ex vivo or in vitro. In particular, the step of contacting a test agent with P2X7R or with a cell or tissue that comprises P2X7R may be carried out in vivo, ex vivo or in vitro. The screening methods of the invention may be carried out in a cell-based or a cell-free system. For example, the screening method of the invention may comprise a step of contacting a cell or tissue comprising P2X7R with a test agent and determining whether the presence of the test agent leads to an increase in the amount or activity of P2X7R in the cell or tissue.

For example, the ability of a test agent to increase the activity or expression of P2X7R may be tested in a host cell or tissue that expresses P2X7R. For example, the amount or activity of P2X7R may be assessed in vitro, in vivo or ex vivo in the kidney or in tissue or cells derived from the kidney, in tissue or cells from another organ that expresses P2X7R, such as macrophages or monocytes.

In such a cell-based assay, the P2X7R and/or the test agent may be endogenous to the host cell or tissue, may be introduced into a host cell or tissue, may be introduced into the host cell or tissue by causing or allowing the expression of an expression construct or vector or may be introduced into the host cell or tissue by stimulating or activating expression from an endogenous gene in the cell.

In such a cell-based method, the amount of P2X7R may be assessed in the presence or absence of a test agent in order to determine whether the agent is altering the amount of P2X7R in the cell or tissue, such as through regulation of P2X7R expression in the cell or tissue or through stabilisation of P2X7R protein within the cell or tissue. The presence of a higher P2X7R activity (e.g. a increased amount of signalling via P2X7R) or an increased amount of P2X7R within the cell or tissue in the presence of the test agent indicates that the test agent may be a suitable agonist of P2X7R for use in accordance with the present invention in the treatment of an individual having kidney injury.

In one embodiment, such a cell based assay may be carried out in vitro or ex vivo on cells or tissue deriving from the patient to be treated. It may therefore be determined whether or not the test agent is capable of increasing the activity or amount of P2X7R in the cells or tissue of that subject. For example, such a method may be carried out on a sample of cells or tissue from the kidney of the patient.

A method of the invention may use a cell-free assay. For example, the P2X7R may be present in a cell-free environment. A suitable cell-free assay may be carried out in a cell extract. For example, the contacting steps of the methods of the invention may be carried out in extracts obtained from cells that may express, produce or otherwise contain P2X7R and/or a test agent. A cell-free system comprising P2X7R may be incubated with the other components of the methods of the invention such a test agent.

In such a cell-free method, the amount of P2X7R may be assessed in the presence or absence of a test agent in order to determine whether the agent is altering the amount of P2X7R in the cell or tissue, such as through stabilisation of P2X7R protein. In either case, the presence of a higher P2X7R activity or an increased amount of P2X7R in the presence of the test agent indicates that the test agent may be a suitable agonist of P2X7R for use in accordance with the present invention in the treatment of an individual having kidney injury.

The contacting step(s) of the method of the invention may comprise incubation of the various components. Such incubations may be performed at any suitable temperature, typically between 4° C. and 40° C. Incubation periods may be selected for optimum activity, but may also be optimized to facilitate rapid high-throughput screening. Following the contact and optional incubation steps, the subject methods may further include a washing step to remove unbound components, where such a washing step is generally employed when required to remove label that would give rise to a background signal during detection, such as radioactive or fluorescently labelled non-specifically bound components.

Incubation in cell or cell-free assay systems may be performed in a microtiter plate (e.g. a 96-well plate or other microwell plate). Further, incubation may be performed in an automated fashion (e.g. for high-throughput screening).

A screening method of the invention may be carried out in vivo. For example, a screening method may be carried out in an animal model. The animal model may be a model of cirrhosis. The animal model may be a model of kidney injury such as acute kidney injury. In such an in vivo model, the effects of a test agent may be assessed in the kidney, or in other organs, cells or tissues that express P2X7R. Preferably, the animal is a non-human animal such as a rat. For example, a screening method may be carried out in a bile duct-ligated rat model or a bile duct ligated rat treated with LPS such as a rat described in the Examples. Such a model may be suitable for identifying agents capable of increasing P2X7R levels. Accordingly, the screening method of the present invention may comprise the step of administering a test agent to a bile duct ligated rat and determining whether the presence of the test agent leads to an increase in the amount or activity of P2X7R in the kidney or other organs, cells or tissues of the rat as discussed above. For example, immunohistochemistry or western blotting may be used to assess the expression of P2X7R in tissues such as the kidney.

Such a model may be used to assess the in vivo effects of a test agent. For example, such a model may be used to assess whether the test agent is capable of increasing the activity or amount of P2X7R in vivo. In such a method, the amount of P2X7R may be assessed and/or the activity of P2X7R, such as signalling by P2X7R, may be assessed.

Such a model may be used to assess the effects of the test agent on the kidney of the model animal.

For example, urine may be collected from the model animal to assess urinary biomarkers of kidney injury such as levels of NGAL (neutrophil gelatinase-associated lipocalin), an early biomarker for renal injury.

Blood may be collected from the model animal to assess other biomarkers of kidney injury such as serum creatinine levels. NGAL may also be measured in the blood or plasma. Blood samples from the model animal may also be used for assessment of levels of endotoxin or cytokines such as TNFα and for other biochemistry.

An in vivo model may also be used to determine whether the test agent has any unwanted side effects. For example, a method of the invention may compare the effects of a test agent on P2X7R with its effects on other receptors in order to determine whether the test agent is specific.

In an in vivo model as described herein, or an in vitro model such as a cell-based or cell-free assay model as described herein, the effects of a test agent on P2X7R may be compared with the effects of the same agent on other purinergic receptors. As discussed above, a preferred P2X7R agonist for use in a method of treatment as described herein may be an agent that agonises P2X7R, but that does not agonise other purinergic receptors. The screening methods of the invention may thus include an additional step of assessing whether the test agent has any effect on the activity or amount of one or more other purinergic receptors such as one or more purinergic receptors that are not P2X7R. In such a method, a test agent may be identified as a suitable P2X7R agonist if it is found to increase the activity or amount of P2X7R, but not to increase, not to significantly increase, not to alter, or not to significantly alter, the activity or amount of one or more other purinergic receptors in the same assay. A test agent may be identified as a suitable P2X7R agonist if it meets any of the requirements discussed above for a selective P2X7R agonist of the present invention. For example, a suitable P2X7R agonist may not increase the activity of one or more other purinergic receptors, or may increase the activity of other purinergic receptor(s) to a lesser extent, such as a lower percentage increase, such as less than 20%, less than 10%, less than 5%, less than 2%, less than 1% or less than 0.5% when compared to its effect on P2X7R. The one or more other purinergic receptors may be other P2 receptors or other P2X receptors.

Where the assay is carried out in vivo, for example in a bile duct ligated rat model as described herein, such as a BDL rat treated with LPS, such a method may comprise comparing the amount or activity of P2X7R in the kidney or other organs of the test animal in the presence or absence of the test agent. An observation that the level or activity of P2X7R is increased in the kidney or other organs of animals treated with the test agent suggests that the test agent may be a suitable agonist of P2X7R. A further finding that treatment with the same test agent does not significantly increase or alter the levels or activity of one or more other purinergic receptors, such as other P2 or P2X receptors, may further indicate that the test agent is a suitable specific agonist of P2X7R that may be used in the methods of treatment described herein. A finding that intra-renal treatment with the test agent in a BDL+LPS animal reduces levels of plasma or serum creatinine may indicate that the agent is a suitable P2X7R agonist.

In the screening methods described herein, the presence of a higher P2X7R activity or an increased amount of P2X7R in the presence of the test agent indicates that the test agent may be a suitable agonist of P2X7R for use in accordance with the present invention to treat an individual having kidney injury, such as acute kidney injury, such as acute kidney injury associated with cirrhosis.

A test agent that is an agonist of P2X7R may result in an increase in P2X7R activity (e.g. signalling) or levels of at least 5%, at least 10%, at least 25%, at least 50%, at least 60%, at least 75%, or at least 85% or more in the presence of the test agent compared to in the absence of the test agent.

Levels or amounts of P2X7R may be measured by assessing expression of the P2X7R gene. Gene expression may be assessed by looking at mRNA production or levels or at protein production or levels. Expression products such as mRNA and proteins may be identified or quantified by methods known in the art. Such methods may utilise hybridisation to specifically identify the mRNA of interest. For example such methods may involve PCR or real-time PCR approaches. Methods to identify or quantify a protein of interest may involve the use of antibodies that bind that protein. For example, such methods may involve western blotting. Regulation of P2X7R gene expression may be compared in the presence and absence of a test agent. Thus test agents can be identified that increase P2X7R gene expression compared to the level seen in the absence of the test agent. Such test agents may be suitable agonists of P2X7R in accordance with the invention.

The effects of a test agent may be assessed by assessing the effects of that agent on a cell that expresses P2X7R. The specificity of the agent may be assessed in a similar way, by assessing morphometry of the receptor on several cell types which express either only P2X7R, or other receptors that are not P2X7R, such as other purinergic receptors, and testing for downstream signals to determine specificity. Such experiments may be carried out using cell types that are known to express the various purinergic receptor types. Such experiments may be carried out using cells that have been engineered to contain or express one or more purinergic receptor types, such as P2X7R that would not naturally be expressed by such cells.

### Pharmaceutical Formulations

A suitable P2X7R agonist as described herein is typically formulated for administration with a pharmaceutically acceptable carrier or diluent. The agonist may be any agonist as defined herein including any agonist identified by a screening method of the invention. The agonist may thus be formulated as a medicament with a standard pharmaceutically acceptable carrier(s) and/or excipient(s) as is routine in the pharmaceutical art. The exact nature of the formulation will depend upon several factors including the desired route of administration. Typically, the agonist may be formulated for oral, intravenous, intragastric, intravascular, intrarenal or intraperitoneal administration.

The pharmaceutical carrier or diluent may be, for example, an isotonic solution such as physiological saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with ornithine and at least one of phenylacetate and phenylbutyrate, a pharmaceutically acceptable carrier, e.g. sterile water, olive oil, ethyl oleate, glycols, e.g. propylene glycol, and if desired, a suitable amount of lidocaine hydrochloride.

Where the agonist to be administered is a nucleic acid molecule, for example where the agonist is in the form of an expression vector, certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules.

A pharmaceutical formulation in accordance with the present invention may further comprise one or more additional therapeutic agents. For example, the formulation may comprise one or more P2X7R agonists as defined herein. The formulation may comprise one or more P2X7R agonists as described here and also one or more additional therapeutic agents. Preferably the additional therapeutic agent(s) are agents which will assist in the treatment or prophylaxis of the individual to be treated. For example, one or more agents that are effective at treating or preventing kidney injury may be administered as part of a formulation as described herein. One or more agents that are effective at treating an underlying condition or symptom thereof in the patient may be administered as part of a formulation as described herein. For example, where the patient has cirrhosis, one or more agents effective at treating or ameliorating the cirrhosis may be administered in combination with an agonist of the present invention. Where the patient has sepsis or cancer, one or more agents effective at treating or ameliorating the sepsis or cancer may be administered in combination with an agonist of the present invention. Where the patient has undergone surgery or trauma or is expected to undergo surgery or trauma, one or more agents effective at treating or preventing the effects of such surgery or trauma may be administered in combination with an agonist of the present invention.

### Treatment

The present invention provides methods for the treatment of individuals having kidney injury, particularly for the treatment of symptoms and conditions associated with or resulting from kidney injury. Accordingly, the invention provides a method of treating an individual having kidney injury comprising administering to said subject an agonist of P2X7R. The present invention also provides methods for the of individuals who are predisposed to or at risk of kidney injury, particularly for the prevention of symptoms and conditions associated with or resulting from kidney injury. Accordingly, the invention provides a method of treating an individual at risk of kidney injury comprising administering to said subject an agonist of P2X7R. Similarly, an agonist of P2X7R may be provided for use in a method of treating an individual having, or at risk of, kidney injury. Also provided is the use of an agonist of P2X7R in the manufacture of a medicament for use in the treatment of an individual having, or at risk of, kidney injury. In any of the methods and uses described herein, the kidney injury may be acute kidney injury.

The agonist may be any agonist as described herein including any agonist identified by a screening method of the invention. The agonist may be provided in a formulation as described herein. An agonist of P2X7R as described herein is thus administered to a subject in order to treat or prevent kidney injury, or particular symptoms or conditions associated with kidney injury in the subject. An agonist of P2X7R as described herein can thus be administered to improve the condition of a subject, for example a subject suffering from kidney injury. An agonist of P2X7R as described herein may be administered to alleviate the symptoms of a subject, for example the symptoms associated with kidney injury. An agonist of P2X7R as described herein may be administered to combat or delay the onset of kidney injury or any symptom associated therewith. The invention can therefore prevent the medical consequences of kidney injury. The individual may be at risk of kidney injury, for example due to chronic liver disease such as cirrhosis. The methods described herein may be used to prevent or delay the onset of kidney injury such as acute kidney injury, in such a patient, such as a patient having cirrhosis. Use of an agonist of P2X7R as described herein may thus extend the life of a patient with kidney injury or a patient having liver disease.

Where used herein, the term kidney injury includes acute kidney injury. Thus, the kidney injury as described herein may be or may comprise acute kidney injury.

The kidney injury may be associated with liver disease or liver injury, For example, the individual to be treated may be suffering from liver failure, such as acute liver failure (ALF) or acute on chronic liver failure (ACLF). The individual may be suffering from chronic liver disease such as cirrhosis. The individual may be suffering from alcoholic liver disease (e.g. which may include hepatitis) or non-alcoholic fatty liver disease. The individual may have cirrhosis and also be at risk of acute kidney injury, such as a patient having cirrhosis and superimposed bacterial infection or gastrointestinal bleeding or a cirrhotic patient that is due to undergo surgery.

The individual to be treated may be suffering from any of these liver diseases in combination with kidney injury such as acute kidney injury. The invention may be used to treat kidney injury such as acute kidney injury in a patient that is suffering from a liver disease as described above, such as cirrhosis, acute on chronic liver failure or acute liver failure.

The individual to be treated may be at increased risk of kidney injury such as acute kidney injury as a consequence of their suffering from one or more of these liver diseases. The invention may be used to prevent kidney injury such as acute kidney injury in a patient that is suffering from a liver disease as described above, such as cirrhosis, acute on chronic liver failure or acute liver failure.

The individual may be suffering from, or at risk of, one or more symptoms or conditions caused by or associated with liver disease or cirrhosis. Any one or more of these conditions or symptoms may be treated or prevented in accordance with the present invention. For example, the individual may be suffering from, or at risk of, one or more of the following as a result of their liver disease or cirrhosis: peripheral vasodilation (e.g. associated with normal, reduced or increased cardiac output); splanchnic vasodilation (e.g associated with normal, reduced or increased cardiac output); reduced cardiac output, portal hypertension, reduced mean arterial pressure, reduced hepatic arterial blood flow, increased intrahepatic resistance, increased plasma ammonia, increased brain water, increased plasma creatinine, renal dysfunction, hepato-renal dysfunction, increased plasma lactate, increased plasma alanine and/or aspartate aminotransferase, alcoholic liver disease, non-alcoholic fatty liver disease.

The kidney injury may not be associated with liver disease or liver injury. The kidney injury may be associated with different conditions or circumstances such as sepsis, trauma, surgery or cancer. For example, the individual to be treated may be suffering from sepsis or cancer, such as cancer of the kidney. The individual may have suffered trauma, such as trauma effecting the kidney or trauma causing injury or acute injury to the kidney. The individual may have undergone surgery, or may be scheduled to undergo surgery, such as surgery that might impact the kidney, such as surgery on the kidney or associated organs or tissues.

The individual to be treated may be suffering from any of these diseases or trauma or surgery in combination with kidney injury such as acute kidney injury. The invention may be used to treat kidney injury such as acute kidney injury in a patient that is suffering from sepsis or cancer, that has undergone trauma or that has undergone or will undergo surgery.

The individual to be treated may be at increased risk of kidney injury such as acute kidney injury as a consequence of their suffering from sepsis and/or cancer and/or one of the liver diseases discussed above and/or trauma and/or surgery as discussed above. The invention may be used to prevent kidney injury such as acute kidney injury in an individual that is suffering from such sepsis and/or cancer and/or liver disease, an individual suffering from trauma or an individual who has had, or who will have surgery as discussed above.

As described herein, the agonist of P2X7R may lead to increased expression and/or increased levels of P2X7R in the kidney of the subject. For example, the agonist may be an agent that promotes transcription of P2X7R in cells of the subject.

As described herein, the agonist of P2X7R may lead to increased activity of P2X7R in the kidney of the individual.

The subject is treated with an agonist of P2X7R as described herein. As described above, the agonist of P2X7R may be administered alone or in the form of a pharmaceutical formulation. The formulation may comprise one or more agonists of P2X7R and may comprise one or more additional therapeutic or prophylactic agents.

Two or more different P2X7R agonists as described herein may be used in combination to treat a subject. The two or more agonists may be administered together, in a single formulation, at the same time, in two or more separate formulations, or separately or sequentially as part of a combined administration regimen.

An agonist or formulation of the invention may be administered by any suitable route. Preferably it is administered by oral, intravenous, intragastric, intrarenal, intraperitoneal or intravascular routes. The agonist or formulation may be administered directly to the kidney(s) of the subject.

The agonist is administered in a therapeutically effective amount. A suitable dose of an agonist of the invention can be determined according to various parameters such as the age, weight and condition of the subject to be treated; the type and severity of the disease; the route of administration; and the required regimen. A suitable dose can be determined for an individual agonist. For example, for some agonists a typical dose may be in the order of from 1 mg/kg/day to 30 g/kg/day. A physician will be able to determine the required dosage of agonist and for any particular subject.

The present invention is broadly applicable to therapeutic methods and is relevant to the development of prophylactic and/or therapeutic treatments. It is to be appreciated that all references herein to treatment include curative, palliative and prophylactic treatment.

Prophylaxis or therapy includes but is not limited to eliciting an effective increase in P2X7R amount, function or activity in order to cause a reduction in one or more symptoms or conditions associated with, or resulting from, kidney injury, such as acute kidney injury associated with cirrhosis. The symptoms or conditions may be, for example, any of those discussed above. For example, prophylaxis or therapy may result in: decreased urinary NGAL, decreased creatinine levels, decreased markers of inflammation such as IL-1β and IL-18. Prophylaxis or therapy may result in the prevention or delay of such symptoms, such as the prevention or delay of kidney injury in an individual at risk of such kidney injury, such as an individual with cirrhosis. Prophylaxis or therapy may result in the maintenance of a particular level of symptoms in a patient where symptoms have been increasing or are expected to increase as a result of the kidney injury and/or cirrhosis. Prophylaxis or therapy may result in the maintenance of a particular level of symptoms in a patient where such factors have been increasing or in which such factors are expected to increase as a result of the kidney injury and/or cirrhosis. Prophylaxis or therapy may result in such changes in symptoms or conditions in such an individual changing at a reduced rate compared to the changes that would have been seen or would have been expected in the absence of such treatment.

Prophylaxis or therapy may have similar effects in relation to any of the symptoms or consequences of kidney injury or cirrhosis described herein. That is, treatment in accordance with the present invention may lead to a lessening in the severity of such symptoms or consequences, maintenance of an existing level of such symptoms or consequences or a slowing or reduction in the worsening of such symptoms or consequences.

### Patients to be Treated

The present invention relates to the treatment of kidney injury such as acute kidney injury in individuals in need thereof. An individual to be treated in accordance with the present invention may therefore have kidney injury such as acute kidney injury or may be at increased risk of kidney injury such as acute kidney injury. For example, the individual may have cirrhosis. The individual may have kidney injury such as acute kidney injury due to causes other than cirrhosis, such as sepsis, trauma, surgery or cancer.

The individual to be treated may have been diagnosed as suffering from cirrhosis, or one or more symptoms or conditions as described herein that may be associated with cirrhosis, for example by any of these methods. The individual to be treated may have been diagnosed as being at risk of cirrhosis or such symptoms or conditions. For example, the individual may have been diagnosed with one or more symptoms that are associated with cirrhosis. For example, the individual to be treated may have liver cirrhosis, alcoholic hepatitis, idiopathic non-cirrhotic portal hypertension, congenital hepatic fibrosis, partial nodular transformation, Budd-Chiari syndrome, portal vein thrombosis, right heart failure or schistosomiasis infection. The methods described herein may be used to prevent or treat kidney injury in a patient having cirrhosis.

An agonist as described herein may be used to prevent or treat kidney injury associated with sepsis, trauma, surgery, cancer or cirrhosis.

The individual to be treated may be identified as having one or more of the following indications: acute on chronic liver failure, acute liver failure, alcoholic liver disease, and non-alcoholic fatty liver disease, non-alcoholic steatohepatitis. An individual suffering from any of these conditions may be treated in accordance with the present invention. The present invention may be used to treat, prevent or ameliorate the effects of kidney injury in a patient having any of these conditions.

The subject may be male or female. Women may be more susceptible to the adverse effects of alcohol than men. Women can develop alcoholic chronic liver disease in a shorter time frame and from smaller amounts of alcohol than men.

The subject to be treated may be a human. The subject to be treated may be a non-human animal. The subject to be treated may be a farm animal for example, a cow or bull, sheep, pig, ox, goat or horse or may be a domestic animal such as a dog or cat. The subject may or may not be an animal model for liver disease. The animal may be any age, but will often be a mature adult subject.

### EXAMPLES

### Example 1

### Effects of P2X7R Antagonist

These experiments utilised an established animal model of cirrhosis, the bile duct ligated (BDL) rat. BDL rats may be generated by methods known in the art. For example, male Sprague-Dawley rats (200-250 g) may be used for this procedure. Following anaesthetisation, a mid-line laparotamy may be performed, the bile duct exposed, triply ligated with 4.0 silk suture, and severed between the second and third ligature. The wound is then closed in layers with absorbable suture, and the animal allowed to recover in a quiet room before being returned to the animal storage facility. Sham animals underwent sham laparotomy. The BDL model was studied 4 weeks after bile duct ligation and liver histology confirmed advanced fibrosis.

LPS (lipopolysaccharide) was given at a dose of 0.5 mg/kg intra-peritoneal. The P2X7R antagonist (A-438079) was given at a dose of 300 μmol/kg 12-hourly 7 days before LPS was given (if applicable). Animals were terminated 3 hours post LPS dose. P2X7RA=P2X7R antagonist.

Urine was collected for assessment of urinary biomarkers of acute kidney injury (AKI). Blood was collected for biochemistry, endotoxin and cytokine analysis. Tissue was collected for histology, immunohistochemistry and western blot. Brain frontal cortex was collected to ascertain brain water. Biochemistry was measured using COBAS integra. Urinary NGAL was measured by ELISA.

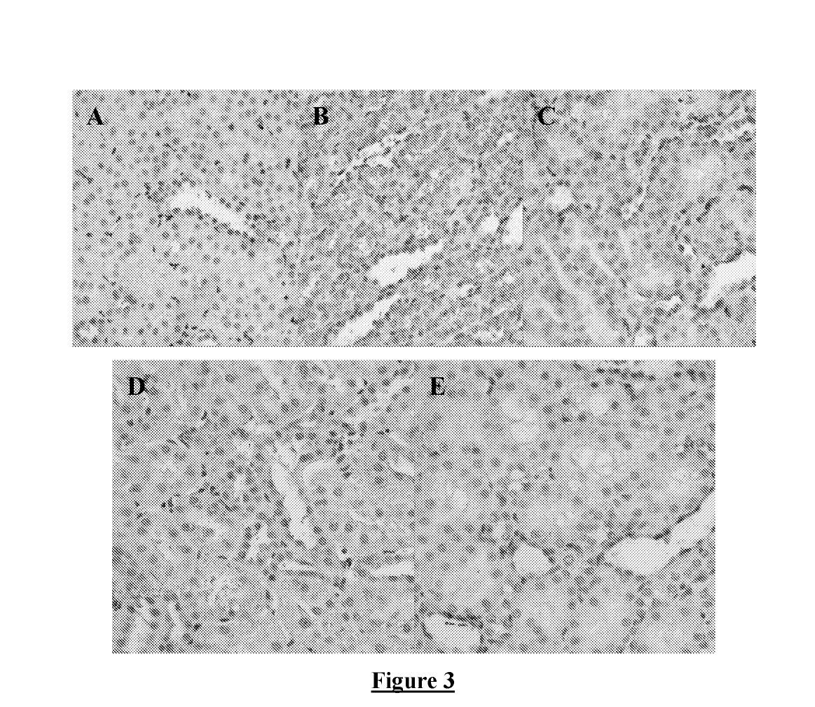

FIG. 3 shows an increased expression in the BDL and BDL+LPS (lipopolysaccharide) treated group compared to sham treated animals. This increase was attenuated in the P2X7R antagonist treated group (n=3 in each group). Sham=sham laparotomy; BDL=bile duct ligated model; SHAM+LPS=sham animals+lipopolysaccharide at 0.5 mg/kg IP; BDL=LPS=LPS ay 0.5 mg/kg IP; BDL+P2X7RA=antagonist A-438079 given IP at 300 μmol/kg 12-hourly for 7 days; BDL+P2X7RA+LPS=A-438079 given as in previous group then LPS given at 0.5 mg/kg IP.

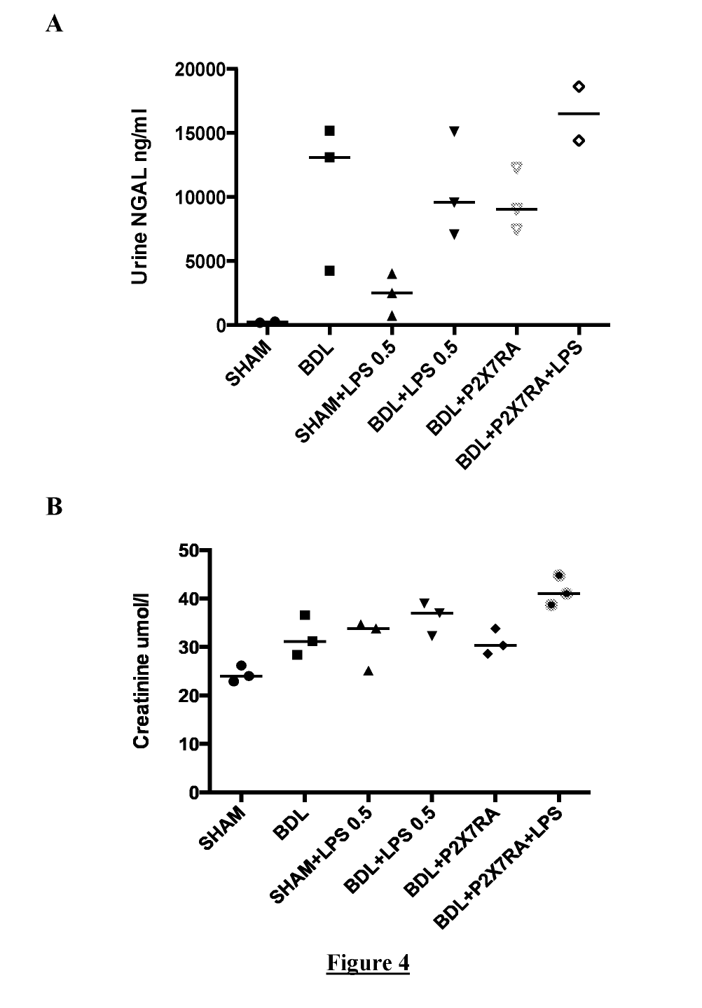

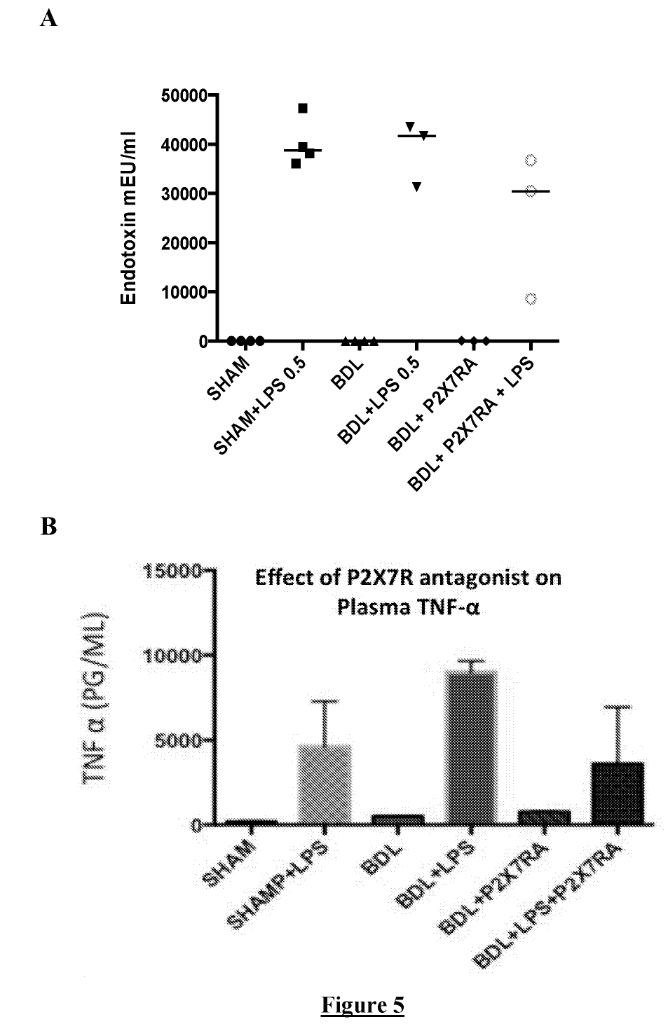

FIG. 4 shows the effects of P2X7R antagonist on biomarkers of acute kidney injury (AKI) in a cirrhotic AKI model. The markers of kidney injury are higher in the BDL+LPS treated with the P2X7R antagonist compared to the other groups.

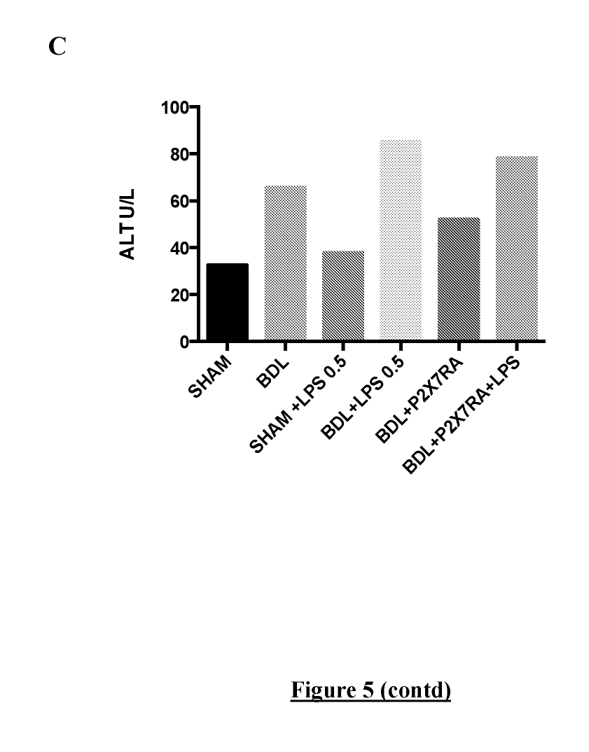

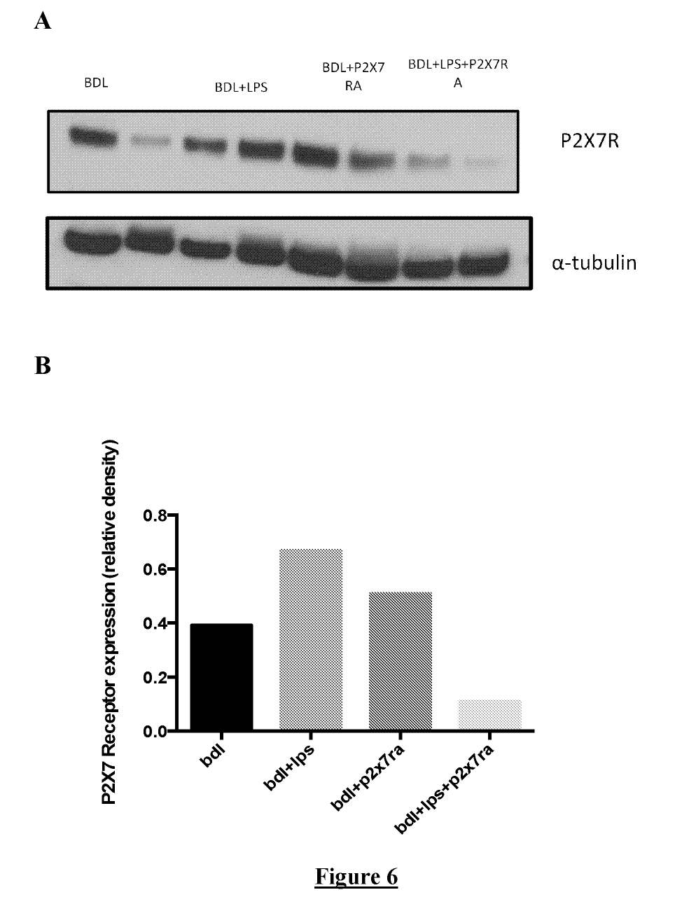

FIGS. 5 and 6 provide further results obtained from the P2X7R antagonist treated animals. FIG. 5 shows the effects of the antagonist on plasma endotoxin, plasma TNFα and ALT (alanine transaminase). FIG. 6 shows the expression of P2X7R in the kidney of the different animal groups.

These results show that the administration of lipopolysaccharide (LPS) to rats with cirrhosis induced by bile duct ligation (BDL), an established model of acute-on chronic liver failure, results in upregulation of the tubular expression of the P2X7R (FIG. 3), which was associated with an acute elevation in biomarkers of AKI (FIG. 4a, 4b). However, this rise was greater in animals treated with a P2X7R antagonist (A-438079) despite the tubular expression of this receptor being attenuated in this particular group of animals. This finding is contrary to that previously described in the literature. It is possible that the P2X7R may be acting as an adaptive protein in the BDL model of cirrhosis and thus blocking the receptor has a detrimental effect on kidney function.

### Example 2

### Effects of P2X7R Agonist

Rats were given a continuous intravenous infusion of LPS at 0.25 mg/kg over 3 hours. The P2X7R agonist BzATP was infused into the renal artery 30 mins before LPS infusion began and continued for the duration of the experiment at a dose of 150 μg/hour.

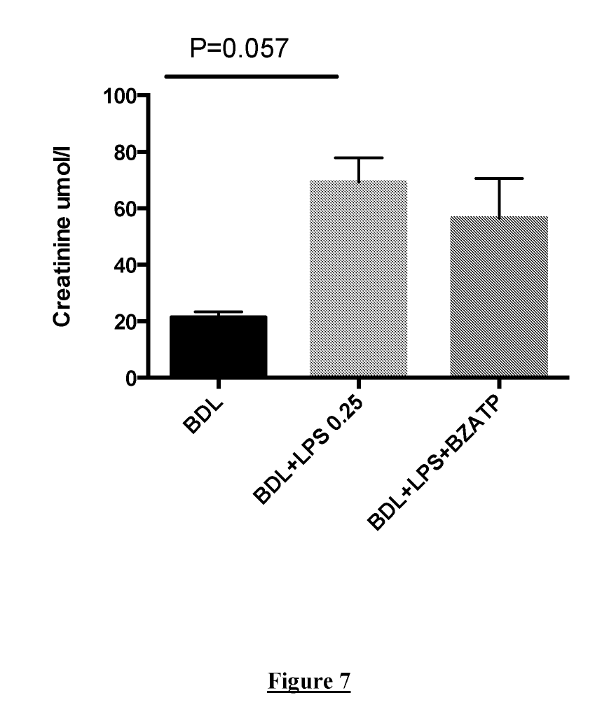

This intra-renal administration of BzATP, a P2X7R agonist was partially protective reducing LPS induced rise in creatinine by 24% (FIG. 7). The mechanism by which receptor inhibition or stimulation modulates kidney injury in this model requires further evaluation.

| | BDL | BDL + LPS | BDL + LPS + BzATP |
|---|---|---|---|
| Creatinine (mean in | 21.37 | 69.18 | 57.93 |
| μmol/L) | | | |
| SD | 3.412 | 17.37 | 12.72 |
| Std Error of Mean | 1.97 | 8.686 | 6.36 |

1. An agonist of the P2X7 receptor for use in a method of preventing or treating kidney injury.

2. An agonist for use according to claim 1 wherein said kidney injury is acute kidney injury.

3. An agonist for use according to claim 1 or 2 wherein the individual to be treated is suffering from liver cirrhosis, acute on chronic liver failure or acute liver failure.

4. An agonist for use according to any one of the preceding claims wherein the individual to be treated:
(a) is suffering from sepsis or cancer;
(b) is suffering from trauma; or
(c) has undergone or will undergo surgery.

5. An agonist for use according to any one of the preceding claims, wherein said method is to prevent the occurrence of acute kidney injury or to treat an episode of acute kidney injury.

6. An agonist for use according to any one of the preceding claims wherein said agonist leads to:
(a) increased expression of P2X7 receptor in the kidney of the individual; and/or
(b) increased levels of P2X7 receptor in the kidney of the individual; and/or
(c) increased activity of P2X7 receptor in the kidney of the individual; and/or
(d) increased signalling via P2X7 receptor in the kidney of the individual.

7. An agonist for use according to any one of the preceding claims wherein said agonist is:
(a) a specific agonist of P2X7 receptor;
(b) a selective agonist of P2X7 receptor;
(c) an indirect agonist of P2X7 receptor that acts by increasing extracellular ATP; or
(d) an indirect agonist of P2X7 receptor that increases the amount of P2X7 receptor.

8. An agonist for use according to any one of the previous claims wherein the agonist is selected from benzoyl benzoyl-ATP and human cathelicidin LL-37.

9. A method of treating or preventing kidney injury in an individual in need thereof, said method comprising a step of administering to said individual an agonist of P2X7 receptor.

10. A method of identifying an agent suitable for use in treating or preventing kidney injury, the method comprising determining whether a test agent is capable of increasing the amount or activity of P2X7 receptor, wherein the ability to increase the amount or activity of P2X7 receptor indicates that the compound may be suitable for use in treating or preventing kidney injury.

11. A method according to claim 10 wherein the amount or activity or P2X7 receptor is assessed in
(a) the kidney or cells derived from the kidney; or
(b) another cell or tissue that expresses P2X7 receptor.

12. A method according to claim 10 or 11, comprising the step of administering a test agent to an animal model of cirrhosis and determining whether the presence of the test agent leads to an increase in the amount or activity of P2X7 receptor in the kidney of the animal.

13. A method according to claim 12 wherein the animal model is
(a) a bile duct ligated rat; or
(b) a bile duct ligated rat treated with lipopolysaccharide (LPS).
